(19) United States
(12) Patent Application Publication
(10) Pub. No.: US 2010/0167334 A1
(43) Pub. Date: Jul. 1, 2010

(54) SYSTEMS AND METHODS FOR PROCESSING TISSUE SAMPLES FOR HISTOPATHOLOGY

(75) Inventor: Warren P. Williamson, IV, Loveland, OH (US)

Correspondence Address: WOOD, HERRON & EVANS, LLP, 2700 CAREW TOWER, 441 VINE STREET, CINCINNATI, OH, 45202, US

(73) Assignee: BioPath Automation, L.L.C., Loveland, OH (US)

(21) Appl. No.: 12/647,701

(22) Filed: Dec. 28, 2009

Related U.S. Application Data
(60) Provisional application No. 61/141,324, filed on Dec. 30, 2008.

(51) Int. Cl. C12Q 1/02; G06F 17/00; C12M 1/34; G06K 19/00

(52) U.S. Cl. 435/29; 235/375; 435/287.1; 235/487; 706/54

(57) ABSTRACT

A system for processing tissue biopsy samples during a histopathology process, including a tissue carrier constructed to carry a tissue biopsy sample during at least one step of the histopathology process, a database storing information associated with the tissue biopsy sample and associated with a tissue processing procedure, a machine-readable indicator physically associated with the tissue carrier, the machine-readable indicator including a machine-readable reference identified with the tissue biopsy sample, and an electronic control operative to read the reference, access the information in the database using the reference, and implement at least a portion of the tissue processing procedure in accordance with the accessed information.

This application claims the benefit of pending provisional application Ser. No. 61/141,324, filed on Dec. 30, 2008 (pending), the disclosure of which is hereby fully incorporated by reference herein.

### BACKGROUND

Current patient records contain information relating mostly to patient and doctor identification and insurance and/or payment coverage. When a patient registers at an admitting office, for example, a medical provider's visit record is initiated. Typically, these records are stored in a healthcare information system (HIS) within the hospital records. This record can show billing codes, insurance data, services and materials used during the treatment of the patient. In the case in which a patient requires a surgical biopsy, records must be maintained to track the biopsy tissue sample, sometimes referred to simply as a biopsy.

Currently, patient records are a compilation of paper charts, stickers, bar codes and computer billing records, etc., but this information will not contain specific handling instructions or specific history of the tissue samples themselves. Batching and process parameter selections are made on a sample-by-sample basis or manually batched by rudimentary classifications of samples, such as like tissue type and/or like tissue sample sizes.

At the time of a tissue biopsy harvested from a patient, information required to track the sample(s) is taken. Many different steps are involved and subject to error or loss of information that can be useful in downstream procedures during the histopathological process. Today, a single tracking number is assigned to a biopsy tissue sample taken from a patient. This tracking number may be manually recorded or may be in the form of a bar code on a label or sticker applied to biopsy containers and tissue preparation cassettes. Biopsy containers are used to transport the tissue to a laboratory for histopathological analysis, while cassettes may be used to process tissue for purposes of embedding the tissue sample in paraffin, microtome sectioning and microscope slide preparation.

A review of the individual steps or procedures typically followed for processing diagnostic tissue samples illustrates how many changes in custody occur during different process steps. Each of these changes in custody creates a situation for potential error. However, each of these different process steps also creates an opportunity to tailor tissue specific processing parameters in the downstream steps or procedures. In addition, information specific to the tissue sample is currently not automatically used by machines for steps or procedures in the histopathological process.

Typically, tissue samples are taken or harvested from a patient in a surgical suite by various methods. Samples are placed in biopsy containers which contain buffering and fixing liquids to condition the tissue prior to arrival at the histopathology laboratory. More specifically, the following steps illustrate a typical process:

1. In the surgery suite, date, patient ID, attending physician and specimen type information can be handwritten on each individual sample container and in a surgical record.

2. Transporting the sample to the histopathology lab can be as simple as using an established delivery route within a hospital. In rural areas the sample may travel many miles from a surgery suite to a stand-alone pathology lab or the sample could even be sent by commercial carrier.

3. The sample container must have enough information on it to be able to positively link that container to the patient's surgical record created in step 1.

4. Once the sample is delivered to the pathology lab, the laboratory personnel match the patient's requisitions to the sample containers and sign and date a log book with identical information.

5. After log-in, the histology specimen or sample information is entered into the laboratory records computer database. The database software examines birthdates and names to verify correct patient identification. All cases have a unique accession number assigned to the case. These numbers are generated in successive order as the samples are logged into the histopathology system.

6. The histo-technologist then writes the accession number on the requisition and the sample container. Tissue processing cassettes are labeled accordingly.

7. The pathologist or his/her assistant, grossing in the case, verifies the matching accession number on the requisition, sample container and cassettes.

8. Tissue processing cassettes with tissue samples inside are placed in the tissue processor. After processing to remove fluid from the samples, the tissue samples are embedded in paraffin blocks which prepare them for sectioning in the microtome.

9. During microtomy, which is the step where the paraffin-embedded tissue is sectioned in a microtome, the tissue sample and cassette matriculate into thinly sliced sections which are placed on glass slides. At this point, the case accession number needs to be transferred to each individual slide created from the paraffin block.

10. Each slide is stained and cover slipped. Finished slides are delivered to a pathologist for diagnostic review under a microscope.

11. The pathologist receives each case or slide(s) accompanied by the surgical report, gross-in information and the processing information. The pathologist formulates his/her diagnosis and completes the surgical pathology report.

12. Slides and paraffin blocks are archived in the event a second opinion or second review is required.

The use of RFID tags or chips to track items in industry is well known. Today, some commercial shipments use RFID tags to track a package at various points during shipping to monitor and record the location of the shipment toward its destination. The use of RFID tags has also been suggested at the end of a histopathology process by attaching RFID tags to an appropriate area of a microscope slide. It would be desirable to provide devices, methods and systems for using machine-readable indicators for purposes of more comprehensively controlling the histopathological process and its individual procedures.

### SUMMARY

In a first illustrative embodiment, the invention comprises a system for processing tissue biopsy samples during a histopathology process comprising generally a tissue carrier, a database, a machine-readable indicator physically associated with the tissue carrier, and an electronic control. The tissue carrier is constructed to carry a tissue biopsy sample during steps of the histopathology process. The database stores information associated with the tissue biopsy sample and associated with a tissue processing procedure used during the histopathology process. The machine-readable indicator includes a machine-readable reference identified with the tissue biopsy sample. The electronic control is operative to read the reference physically associated with the tissue carrier, access the information in the database using the reference, and implement at least a portion of the tissue processing procedure in accordance with the accessed information.

The tissue processing device may directly include the electronic control to carry out at least a portion of the tissue processing procedure, for example, performed with that tissue processing device. Alternatively, the electronic control may be separate and even remote from the tissue processing device. The tissue processing device, for example, may be a tissue gross-in device, a tissue processor, a tissue embedding device, a tissue microtomy device, a tissue slide preparation device, or a diagnostic slide reading device. Other types of tissue processing devices may be used in carrying out this invention as well or as alternatives. The electronic control may have various functions, such as updating the information in the database and updating the information stored on the machine-readable indicator, in addition to implementing or controlling at least a portion of the tissue processing procedure by using at least part of the information contained on the machine-readable indicator and/or in a separate database. The machine-readable indicator may comprise various forms. For example, the indicator may be an RFID tag or another electronic memory device. Alternatively, the machine-readable indicator may comprise simpler forms such as a machine-readable digital reference code (DRC). In this form, for example, the digital reference code or DRC may be a simple read-only indicator. More specifically, such a read-only indicator may comprise a bar code, etched code or other indicia that is machine-readable and physically associated with the tissue carrier.

In another embodiment, a system is provided for processing tissue biopsy samples during a histopathology process and generally comprises a tissue carrier, a machine-readable and writable indicator physically associated with the tissue carrier, and an electronic control. In this embodiment, the tissue carrier is again constructed to carry a tissue biopsy sample during at least one step of the histopathology process. The machine-readable and writable indicator is physically associated with the tissue carrier and stores a reference identified to the biopsy tissue sample and further stores information associated with at least one physical attribute of the tissue biopsy sample. This physical attribute may, for example, be one or more of: the tissue size or volume, the tissue type, the suspected disease profile or pathology of the tissue, or any other physical attribute. The electronic control is operative to read the reference and the information stored on the machine-readable and writable indicator and implement at least a portion of the tissue processing procedure in accordance with the accessed information. The electronic control may be further operative to write new information on the machine-readable and writable indicator. The new information may comprise updating the information already stored on the machine-readable and writable indicator. Information stored on the machine-readable and writable indicator may be identical to at least a portion of information stored in a separate database. When a separate database is also used as part of the system in addition to the machine-readable and writable indicator, the tissue processing procedure may be implemented by the electronic control through interaction of the electronic control with both the machine-readable and writable indicator as well as the database. This database may be part of a remote computer system or may be part of the electronic control. In various other embodiments a database may be included in the electronics of the machine-readable indicator. The information on the machine-readable and writable indicator may alternatively be at least be partially different than the information stored in the separate database. The system may further comprise a tissue processing device that includes the electronic control and is operative to carry out at least a portion of the tissue processing procedure. Still other attributes of this system may be incorporated as generally discussed herein.

In another embodiment of the invention, a system for embedding tissue biopsy samples during a histopathology process is provided and generally comprises a tissue carrier as discussed above, as well as a database, machine-readable indicator, and an electronic control as discussed above. This system further includes an embedding device operative to embed the tissue biopsy sample for subsequent microtomy. In this embodiment, the electronic control accesses the information in the database and implements at least a portion of the embedding procedure conducted by the embedding device in accordance with the information. For example, the information stored in the database may be used to direct the embedding device to place the tissue biopsy sample, with or without the tissue carrier, into one of at least two different molds for receiving the embedding material. This may include using a sectionable carrier that holds the tissue biopsy sample and is placed in the mold with the tissue sample. The database may be remote from the tissue carrier, or may be on the machine-readable indicator itself. In the latter case, the information stored in the machine-readable indicator may be used to direct the embedding device to place the tissue biopsy sample into one of at least two different molds for receiving the embedding material. This, for example, can allow the use of a small mold and a large mold, depending on the size of the tissue carrier. The information stored in the database may also or alternatively be used to direct the embedding device to heat and/or cool embedding material used for embedding the tissue biopsy sample. Again, this information may also or alternatively be stored more directly in the machine-readable indicator. The information may also or alternatively be used to select a type of embedding material from multiple types of material for embedding the tissue biopsy sample, for example, according to tissue type.

In another embodiment, a system for processing tissue biopsy samples during a histopathology process includes a tissue carrier, a tissue processor, a database, a machine-readable indicator, and an electronic control. The tissue carrier, database, machine-readable indicator, and electronic control may be generally as described above. The tissue processor is operative to subject the tissue biopsy sample to a procedure for enabling subsequent embedding of the tissue biopsy sample. In conventional tissue processors, this includes the use of chemical reagents to remove fluids from the tissue and replace those fluids with a paraffin-like substance thereby preparing the tissue sample for full embedding in paraffin. Other tissue processors may use, for example, microwave technology for purposes of removing fluids from the tissue. In a system constructed in accordance with this embodiment, the electronic control accesses the information in the database and implements at least a portion of the procedure using the tissue processor in accordance with the accessed information. The information, for example, may direct the tissue processor in accordance with: chemical reagents used during the process cycles, cycle times of the processor, cycle temperatures of the processor, and the like.

In another embodiment, a system is provided for carrying out at least a portion of a histopathology process. The system generally includes a tissue carrier constructed to carry a tissue biopsy sample, a machine-readable indicator physically associated with the tissue carrier, and a database. The machine-readable indicator provides a machine-readable reference identified with the tissue biopsy sample. The database stores a tissue sample record associated with the reference of the machine-readable indicator. The information in the tissue sample record is usable to assist with the performance of at least one step in the histopathology process of the biopsy tissue sample. The information in the tissue sample record may be used to assist in the performance, for example, of at least one of: processing of the tissue biopsy sample prior to embedding of the sample, embedding of the tissue biopsy sample, microtomy slide preparation of the sample, staining of the sample on a microscope slide or preparation of a final pathology report on the tissue biopsy sample.

In another embodiment, a system is provided for processing tissue biopsy samples during a histopathology process and generally comprises a plurality of tissue carriers, a machine operative to carry out at least one procedure during the histopathology process, a database, a plurality of machine-readable indicators respectively physically associated with the tissue carriers, and an electronic control. In this embodiment, each of the plurality of tissue carriers is constructed to carry a tissue biopsy sample during the histopathology process. Each machine-readable indicator includes a machine-readable reference identified with the tissue biopsy sample associated with the corresponding tissue carrier. After accessing the information in the database using the unique references, the electronic control implements at least a portion of the procedure based on like information stored with respect to the different references. Various procedures may be implemented using the system. For example, the electronic control could be used to prevent at least one operation within the procedure from being performed on at least one of the tissue biopsy samples based on the like information. The electronic control may also be programmed to cause at least one operation within the procedure to be performed on at least one of the tissue biopsy samples based on the like information.

The invention also contemplates various methods. For example, in one embodiment, a method of processing tissue biopsy samples during a histopathology process includes: reading a machine-readable indicator physically associated with a tissue carrier to obtain a reference associated with a tissue biopsy sample carried by the carrier; accessing information in a database associated with the reference obtained from the machine-readable indicator; and performing at least one procedure during the histopathology process using the information. In one alternative, a tissue processing device may include a reader and the reader may be used to read the machine-readable indicator. The method may further comprise performing the procedure with the tissue processing device. The tissue processing device, for example, may comprise a tissue gross-in device, a tissue processor, a tissue embedding device, a tissue microtomy device, a tissue slide preparation device, or a diagnostic slide reading device, or any other device used during the histopathology process.

Another method of processing tissue biopsy samples during a histopathology process comprises reading a machine-readable indicator physically associated with a tissue carrier to obtain information associated with the tissue biopsy sample carried by the tissue carrier, and performing at least one procedure during the histopathology process using the information. The method may include the use of a tissue processing device including a reader and the reader may be used to read the machine-readable indicator. The method may further comprise performing the procedure with the tissue processing device. The tissue processing device may be any one of those described previously, or another device.

As illustrative examples, the methods of this invention may perform at least one of the following steps: a) using the information to select a parameter of a tissue gross-in procedure for recording information on the tissue biopsy sample; b) using the information to select a parameter of a tissue fixation procedure for preparing the tissue biopsy sample for embedding; c) using the information to select a parameter of a tissue embedding procedure for preparing the tissue biopsy sample for microtomy and microscope slide preparation; and d) using the information to select a parameter of a microtomy procedure for preparing microscope slides of the tissue biopsy sample.

Another method of processing tissue biopsy samples during a histopathology process includes reading a machine-readable indicator physically associated with a tissue carrier to obtain information associated with the tissue biopsy sample carried by the tissue carrier, accessing an electronic tissue sample record based at least partially on the information, and controlling at least one procedure during the histopathology process using the access information, with the procedure selected from one or more of the following: tissue fixation, tissue embedding, microtomy, microscope slide preparation, microscope slide staining, and preparation of a pathology report.

The methods of this invention may include updating the information in the electronic tissue sample record with information for use in subsequent procedures of the histopathology process. Controlling the at least one procedure may further comprise making inclusionary or exclusionary batch decisions based on a comparison of like or similar parameters associated with different tissue carriers and/or different tissue biopsy samples. The information or reference, such as a DRC, contained on the machine-readable indicator may include a digital reference code associated with at least one of the tissue biopsy sample or the tissue carrier, and the electronic tissue sample record may at least partially be stored in a database remote from the machine-readable indicator. The electronic tissue sample record may at least partially be contained in a database stored in a control of a device used in the histopathology process itself. For example, any of the devices used during processing of the tissue as mentioned above may include an electronic control having the mentioned database. Alternatively, or in addition, the electronic tissue sample record may at least partially be stored on the machine-readable indicator. In other embodiments, the machine-readable indicator only contains a digital reference code and other information of the electronic tissue sample record is stored in a database remote from the machine readable indicator.

Various additional features and advantages of the invention will be recognized by further review of this disclosure and, in particular, the additional detail provided in the description of the embodiments below.

### BRIEF DESCRIPTION OF THE DRAWINGS

FIG. 1 is a schematic illustration of a patient admission procedure in accordance with an illustrative embodiment of the invention.

FIG. 2 is a schematic illustration of a biopsy harvesting procedure or step subsequent to the patient admission procedure.

FIG. 3 is a perspective view of a gross-in center or apparatus used after the biopsy sample is harvested from the patient.

FIG. 4 is a perspective view of a tissue processing machine or tissue processor used after the tissue sample gross-in procedure.

FIG. 5 is a perspective view of a tissue embedding center or machine used after the tissue processing procedure.

FIG. 6 is a perspective view of apparatus used during microtomy procedure subsequent to the tissue embedding procedure.

FIG. 7 is a schematic elevational view of an alternative, automated microtomy apparatus and slide preparation center or device.

FIG. 8 is a perspective view of a slide staining center or device.

FIG. 9 is a schematic view of the pathology review and report procedure.

### DESCRIPTION OF VARIOUS ILLUSTRATIVE EMBODIMENTS OF THE INVENTION

Various embodiments of this invention generally comprise devices, systems and methods that create, record and utilize information about a biopsy or other tissue specimen that can be used to customize the histopathology tissue processing parameters for that specific sample. A digital record, referred to herein as a Tissue Sample Record or TSR, is initiated at the time of patient registration. The digital record or TSR is supplemented or built upon to contain data directly concerning the tissue sample or more generally associated with the sample, such as data concerning processing the tissue sample as the sample continues through the histopathology system. Information associated with the patient's tissue sample is added to the digital record or TSR, such as biopsy procedure records, a histopathology report and a diagnosis, as well as data about the tissue sample that improves the processing and diagnostics of the sample. Of course, the principles of this invention will apply to the processing of many samples at one time.

The TSR may include tissue specific processing instructions that accompany the sample. The TSR prevents losing track of a patient's samples as may occur when multiple records are used and subsequently become disconnected. In addition, lab processing equipment can use the TSR to make multivariable analyses for the specific processing parameters of each tissue sample. This invention can enable a progressively more intelligent, comprehensive and useful record to be built and used in steps or procedures of the histopathologic process. This does not merely involve the sequential tracking of an individual item such as a tissue sample. The invention can enable information about each individual sample to be made available to one or more automated machines for automated decision-making in the tissue handling, processing and slide preparation process by continually updating a database of information (i.e., the TSR) about each tissue sample and its requirements for the optimal processing and diagnostic outcome. In addition, this invention allows for automated machines in the overall histopathologic process to communicate information about individual biopsies or batch processes thereby allowing separate machines to accept workflow from and communicate with other machines intelligently.

The invention not only enhances existing tissue tracking systems but allows for new information to be gathered, stored and used in uniquely useful and varied manners for downstream tissue sample processes. New information stored in the digital record or TSR for each tissue sample is available to computer-controlled tissue processing machines. The information may concern records and processes associated with the tissue sample and can enable new processing efficiencies and diagnostic capabilities that were previously impossible or very cumbersome to accomplish with an overworked manual system.

Information recorded in the digital record or TSR may be used to enable tissue specific processes to be customized for such things as tissue type, suspected disease type, embedding material type, etc. Diagnostic preparations may be customized using the information such as by defining special slide stains, different embedding materials, different tissue processing parameters or the like. Additionally, information about the diagnostic report and pathology lab processes may be stored or recorded in the digital record or TSR for reporting and archival proposes.

As mentioned above, in the disclosed system the digital record comprises a dynamic Tissue Sample Record (TSR) that is continuously updated as the biopsy samples travel from procedure to procedure while processing a tissue sample. At multiple points in the histopathologic process more information may be added to the TSR that will allow subsequent steps to increase efficiencies, capabilities and accuracy throughout the process. Several components of a system or method may work together to allow the storing and retrieval of information between the components of the system. Ultimately, this can culminate in a diagnostic report on the tissue sample.

In a more basic form the invention can comprise a series of tissue carriers which have machine-readable indicators physically associated with them. The machine-readable indicator may comprise basic technology such as a bar code or laser etching on the tissue carrier, or more sophisticated read only or read/write memory technology, such as an RFID tag or chip. The machine-readable indicator includes a reference, which may be as simple as a tissue carrier identifying member. The tissue carrier identifying number referred to herein as a Digital Reference Code (DRC) is also stored in a central database and associated with the TSR for the sample carried by the particular tissue carrier. As the database is continually updated with information about the tissue sample, the TSR becomes dynamic and read/writeable. When the TSR is accessible by the controls of machines used in the overall process, opportunities for improved individual sample preparation becomes possible.

By continually updating the TSR with information concerning the previous step or procedure in the process, the TSR becomes more useful downstream as the sample progresses towards a final diagnostic report.

The following components or steps of the histopathology process can in part or whole be upgraded to allow data storage usage and updating from such a system. For example, the following steps may be involved:

1. Patient admitting: Initiation of patient record and TSR if a biopsy is part of the procedure.

2. Surgery tissue harvest: TSR updated with, for example, type of suspected disease, special handling procedures, staining requirements for microscope slide preparation, and other parameters for suspected diagnosis.

3. Tissue transport to the lab: In preparation, TSR updated with, e.g., number of tissue samples, type of fixing solution and time/date of initial immersion of tissue sample(s) in fixing solution.

4. Tissue gross-in at pathology lab orientation: TSR updated with, e.g., requirements of tissue samples, digital records (e.g., digital photographs) of tissue samples before grossing, special tissue processing requirements identified at this step.

5. Tissue processing with solvents/chemicals, microwave, etc.: In preparation, TSR updated with, e.g., specific tissue processing parameters, such as reagents used, cycle times and temperatures.

6. Tissue embedding: In preparation, TSR updated with, e.g., cassette type for base mold selection size to minimize embedding material (e.g., paraffin), paraffin type, and temperature exposure.

7. Tissue microtomy and slide preparation: In preparation, TSR updated with, e.g., tissue section thickness and number of sections prepared for microscope slide preparation and staining.

8. Tissue staining: In preparation, TSR updated with, e.g., tissue stains for specific tissue types or disease states.

9. Pathology diagnosis and report: TSR updated with, e.g., advanced tissue identification and description methods such as ultrasonic imaging, digitally recorded photographs or video and digitally recorded oral descriptions and transcriptions of those descriptions.

10. Tissue blocks slides and report archive: TSR allows easier, more efficient retrieval of archived blocks, much easier to send a digital pathology report to a consulting professional for second opinion.

In general there are three main features utilizing devices, systems and methods to read/write data and enable enhanced processes from that data:

1. The Tissue Carrier—this can take the form of any mechanical device(s) or support(s) to hold the tissue sample(s) from the initial harvest through gross-in, to processing the tissue in a cassette and then onto microscope slides. The tissue carrier also carries the machine-readable indicator, which may have an associated Digital Reference Code or DRC.

2. The Histopathologic Process Equipment or Machines—gross-in station, tissue processor, automated embedding, microtomy, microscope slide preparation, diagnostics, etc.

3. The Tissue Sample Record (TSR) and updating system—portions of which may be stored within the machine-readable indicator and/or process equipment but the complete TSR desirably resides in one or more databases which can be accessed by tissue process equipment.

By electronically linking the above three features, a more efficient processing system with enhanced capabilities and more complete and accurate records can be maintained for each harvested tissue sample. An electronic or digital TSR may track tissue sample information from the time of tissue harvest to the final pathology diagnostic record and report or during only one or more steps of the overall histopathology process.

RFID tags may be attached to the tissue carrier and used as the machine-readable indicator. Although RFID tags (chips) are attractive for their ability to both read and write information, the majority of the information associated with each tissue sample may be recorded remotely on a server in a database record. The tissue carrier need only have a reference, such as a unique number or code, which can be associated with that patient's surgical record. In fact, it may be more economical to first initiate the predictive and enhanced processing aspects of this invention prior to implementing tissue carriers with both read and write capabilities such as those having physically associated RFID tags. More complete benefits may be realized through the use of RFID technology, or other read/write memory technology, as will be appreciated from the description herein.

An RFID tag consists of various antennae and radiofrequency receiving and transmitting circuitry along with digital storage capacity. The RFID tag or device should be sufficiently embedded or integrated with each tissue carrier to prevent degradation from various tissue processing steps which may include solvents and reagents, microwave radiation from tissue processors, elevated and reduced temperatures, both positive and negative atmospheric pressures, and general shock and vibration. In addition, the RFID tag or other memory device must not lose its memory for periods extending at least 10 years for archival purposes. RFID tags or other memory devices embedded in glass or reagent resistant plastics are desirable.

The Tissue Sample Record or TSR can be established so as to allow process equipment to not only track the individual samples but to be able to recommend further processing enhancements of those samples in downstream procedures or steps in the overall process. Some laboratories will implement technology in various stages. Therefore, it may be necessary to have systems which can read other types of machine-readable indicators such as barcodes which have been previously implemented into the histopathology process. Devices may be incorporated which can read these other types of machine-readable indicators and record them in the Tissue Sample Record (TSR) which may be continuously updated for each step. The digital TSR need not contain all of the required information. Since most hospital systems have database computers which track billing records it is conceivable that a system may utilize software that would be added to these databases.

A specific TSR links to each tissue sample taken from a patient at the time of harvest and is updated with new information through part or all of the histopathology process. As the tissue sample travels through the process, a digital update code can be recorded on a machine-readable indicator physically associated with the tissue carrier (by way of a machine-readable code) or it can be transmitted to the TSR in a centralized data base record remote from the tissue carrier, but referenced to the tissue carrier by way of the DRC. This will update the TSR and keep this information ready and available for the next step in the histopathology process.

A clear, concise record is assured throughout the entire process by linking the chain of custody of a tissue sample with the ability to build a digital database in the form of a TSR for each patient's tissue samples.

The Tissue Sample Record (TSR) is linked to the Digital Reference Code (DRC) which may be, for example, in binary code or Hexadecimal code or any other type of machine-readable code physically associated with the tissue carrier. The DRC may therefore be used as the specific identifier that allows the patient's tissue carrier to be linked to the TSR when the TSR is stored in a database remote from the tissue carrier. The DRC is physically associated with the tissue carrier, through the machine-readable indicator, and is associated with the TSR in the database. Embodiments can be, for example, a bar code label that contains the DRC, a laser-engraved optically readable code (DRC), or a code (DRC) programmed into a digital storage device such as an RFID device. The DRC can be tagged to or associated with any tissue samples harvested from a patient. RFID tags or other machine-readable indicators may be incorporated into or otherwise physically connected with containers, slides or any other tissue carriers used for transporting or holding the tissue sample(s) during the histopathologic process.

The DRC is the link between the tissue carrier and, therefore, the tissue sample(s) in or on the carrier, and the TSR. Due to current RFID chip data storage limitations and cost, it may be advantageous to limit the amount of information that resides physically with the tissue carrier. Cost efficiencies will be important in achieving the least expensive means to associate data with each tissue carrier and, therefore, each tissue sample. As with any digital storage device, the more bits of information stored, the greater the cost. Therefore, a hybrid system may be desirable using a combination of bar codes or similar, simple machine-readable indicators on some carriers and more complex machine-readable indicators such as RFID tags or other read/write memory devices on others. One or more databases may therefore be desirable and cost efficient as a central repository for all of the TSR's. In addition these records can be backed up and archived by information technology staff at the hospital or laboratory.

The system can also include devices and methods to write data to the machine-readable indicator and this data can be used in successive steps of tissue processing by process equipment. This would be useful, for example, if one or more of the pieces of equipment are not electronically connected to the database and cannot retrieve remotely stored data in the database. Readers, such as optical readers, can be used in the machines or equipment to read codes that have been written to a tissue carrier. A certain amount of information can be passed along directly on an RFID chip physically associated or connected with the carrier. Other information can be retrieved from the TSR in one or more databases which is/are remote from the carrier. The DRC will be able to link all of the patient's tissue samples and diagnostic work to a TSR stored in one or more central databases. While today's RFID tags are not practical for handling all the data required to build a TSR, it is conceivable that in the future the entire TSR could be stored on the RFID chip associated with the tissue carrier. In this situation, the reference stored on such a sophisticated machine-readable indicator would comprise more detailed information and/or instructions associated with the process for corresponding tissue sample. It is also possible to assign event codes that are linked to look-up tables. In this manner simple binary code could be associated with some of the TSR. For example, there may only be three types of paraffin that can be used to embed tissue samples, so a two word binary code will cover all three.

As the storage levels of the machine-readable indicators associated with the tissue carriers evolve, a system could be planned allowing for more storage. Until read/write storage such as RFID technology becomes much less costly, simpler systems will be implemented that rely on a hybrid of remote data storage with an efficient manner to access that digital TSR to make the information available to smart tissue process machines. The following shows a migration path of the DRC at three different exemplary levels of sophistication.

The first level would be one in which only a single tissue identifier DRC is used throughout the process for each patient. The single tissue identifier DRC would then be linked at each tissue process station back to the TSR stored in the database(s) of a health care information system. This would require the least amount of memory physically associated with the tissue carriers and no RFID chip or other sophisticated memory device would be needed for the tissue carriers. For example, the DRC could simply be in the form of a unique bar code or other machine readable indicator assigned to a particular tissue sample or samples from a particular patient and applied to the tissue carrier in a suitable manner. This system may still allow limited information to be stored on the tissue carrier to help make smart processing decisions for that specific tissue sample.

In the next scenario or second level a TSR stored in a small amount of read/write memory is available on the machine-readable indicator. In this case, the machine-readable indicatory could comprise an RFID chip or other read/write memory device. This would allow the system to update the machine-readable indicator as it passes through various stations of the histopathologic process thereby adding useful information for downstream customization of tissue processes. One or more of the machines in the process could individually read the TSR for each sample being processed. In this scenario each machine is basically stand-alone in its decision-making processes for the tissue samples currently being run in batches.

In a third level, information about tissue specific tests and process parameters are built up to make a comprehensive dynamic digital file (the TSR) allowing custom processing parameters to be ordered for each sample. Within a hospital system it would then be possible for the machine-readable indicator to simply have a unique number which accesses a unique database record which is updated each time the sample completes a processing step. Each process machine can then query the database by referencing the DRC associated with the tissue carrier to assure each step updates the sample record properly. This is a desirable step in that each change in custody can be tracked to a specific machine or user and a legacy record of the TSR is created as the tissue sample makes its way to the pathologist for diagnosis. In situations where data is too involved to program the RFID chip directly on a tissue carrier a short code can be stored to indicate that the tissue carrier has been programmed for that step. This is also is a way to track the sample through the steps of the entire process and confirming, for example, that the sample has completed each step in proper order. The most sophisticated version of a machine-readable indicator could carry most, if not all, of the TSR, although it would still be desirable to have the TSR electronically stored in one or more other databases remote from the machine-readable indicator, at least for back-up and archival purposes.

The first place where a tissue sample is linked with a TSR is in the surgical suite where the sample is taken from the patient. Each sample or set of samples is placed into a tissue carrier, such as a biopsy container. Each biopsy container would have a machine-readable indicator such as an RFID tag, bar code, laser etched code, etc., which would contain the DRC assigned during the admitting process of the patient or initiation into the laboratory system. The machine-readable indicator could also contain other information that needs to be passed along to the next process step or procedure in the pathology laboratory. This information might include the surgeon's observations as to the type of suspected pathology. This information could be important during later steps associated with processing the sample, such as for specific slide staining for a particular type of cell or disease. Such information could be useful to the pathologist or histotechnologist, or to an automated gross-in system as will be described below. The TSR could also include the name of the doctor that performed the surgery and information specific to the surgical procedure such as the number of biopsy samples placed in the sample container. If instruments are given tracking numbers as has been contemplated in existing hospital tracking systems, the TSR could be updated with information indicating the instrument used to take the biopsy. Specific diseases or tissue types will then be able to receive special tissue processing instructions depending on the tissue type. Some rare diseases may need special slide stains. In this situation tissue can be automatically culled from the standard run batches and be processed on a separate stain setup.

In U.S. Pat. No. 5,817,032, a system was described which included a digital video gross-in. The gross-in station would be located in the pathology laboratory. This is envisioned as an area with a digital camera system under which the samples could be placed. A stereo camera system could be used so that it is possible to record a three-dimensional image depicting the sample prior to its placement in a cassette and prior to chemical/solvent processing, embedding and sectioning. Size reticules in the photograph would allow system software to calculate the overall area of the tissue sample. Ultrasonic imaging may be used to determine the height or volume of a tissue sample. With these parameters recorded, software could make decisions or suggestions about the type of cassette best used to embed the tissue sample. This gross-in station would also have a system to read and write to machine-readable indicators, such as RFID tags, or to create simpler machine-readable indicators such as bar code labels or laser etched codes applied to the tissue carriers. The reading system would read the sample information and specific processing instructions from, for example, the surgical suite by means of the machine-readable indicator on the tissue carrier (e.g., sample container).

The digital video gross-in takes the place of a manual gross-in system whereby the tissue's gross attributes are described into a dictation system by the pathologist upon receipt of the tissue from surgery. With the video gross-in any oral description which may be given is recorded along with the video data and then transposed to digital format such as into .wav files. These files can be stored as voice records in the TSR or these files could then either be transcribed using dictation software, or typed by a transcriptionist. In any case, all of these records could be available for retrieval outside of the hospital. For instance, transcriptionists at remote locations could be given access to specific TSR's for that day's work. Since each TSR has a unique DRC the written transcription can be linked back into the TSR when complete. This information would be digitally added to the TSR. The TSR becomes the central place for depositing all information about a patient's biopsy and diagnostic processing and reporting. As previously stated, the TSR may be electronically stored in one or more remote databases, or on the tissue carrier itself, or using a combination of both.

After the gross-in data is recorded, the pathologist prepares the tissue samples and places them into individual tissue carriers, for example, in the form of cassettes. The cassettes are then each individually serialized with the Digital Reference Code (DRC) unique to each patient. That number or code is also associated with each cassette by means of a machine-readable code associated with a machine-readable indicator physically associated with that cassette. When the pathologist first encounters tissue samples from the patient, the gross-in station will query the sample container to obtain the DRC. The query can be in any format, such as an optically readable code, RF identifier, or even a manual input on a computer keyboard. In addition, a control panel at this gross-in station would allow the pathologist to add instructions, such as from a selection menu or implicate processor code which will update the TSR accordingly for each individual cassette as to how the tissue is to be processed and sectioned further on in the process. This information then may enable the use of subsequent smart-processing and embedding machines which significantly reduce the required human/sample interaction and allows the machine to make automatic processing decisions. Smart processing might contain information such as the tissue type, tissue orientation, or special slide stain required for a specific disease or tissue type. Therefore, samples which have been indicated to have special tissue processing characteristics or requirements can be processed precisely in accordance with those characteristics or requirements.

After the tissue samples are grossed-in, the prepared samples are placed into one or more individual tissue carriers such as biopsy cassettes, and the cassettes are transferred into a tissue processing machine which may use reagents such as solvents and chemicals, or microwaves, etc. to prepare the tissue for embedding and sectioning. The machine-readable indicator of the cassette can be queried at a point prior to entering the processing chamber of the processing machine and sorted according to required process length, reagent parameters, heat or vacuum constraints based on the thickness or size of the sample and tissue type, etc. This is information that was added to the TSR at gross-in of each sample. This will also allow a cross-check on samples to make sure that they are processed properly. For instance, small biopsies are processed in the processing machine at a faster cycle than large biopsy samples. Since small biopsies can be run faster, if the chamber is filled entirely with small biopsies the process can be run on a short cycle. The machine can then make an inclusionary decision to batch samples with like processing requirements and run this batch of samples through one or more parts of the histology process together. Conversely, if one or more samples is/are different than the rest, the machine can exclude the one or more samples from the batch and run the sample(s) separately or divert to another processing machine. By further example, breast tissue has a high fat content and certain reagents clear fat better than others. Such a system can adjust the time and temperature and chemical (reagent) makeup to have tissue-type-specific processing available with a machine equipped to make changes to tissue processing parameters based on the needs of each tissue sample.

A fully automated machine can also sort cassettes by process type and load the appropriate processing chambers with the correct samples for that process sample type. This system would also enable specialized reagents and/or accelerants to be run on specific samples in order to penetrate thick tissues or for other reasons. The system would cull these samples out and prevent them from being processed in a standard batch. The machine would automatically adjust the processing parameters according to the makeup of the samples in the processing chamber.

Once the tissue samples are processed, they may be directed into the automated embedding station or center. Once again, information associated with the machine-readable indicator of each tissue carrier can be read by the automated embedding machine. For instance, if the tissue carrier (e.g., the cassette) includes an RFID tag, or a DRC on a bar code, for example, a database record for that sample can be retrieved for use by the embedding machine and downstream machines. The same may be accomplished for upstream machines in the process. If the machine-readable indicator of the tissue carrier does not contain this information itself, the machine simply queries a remote or local database record for the individual DRC associated with that tissue carrier and calls up the TSR to obtain specific embedding requirements for that sample.

During this step or procedure, the embedding machine will be instructed on how to embed that specific sample on an individual cassette-by-cassette basis. Information in the TSR could also control the particular embedding media which is dispensed for that particular type of biopsy. For instance, different molecular weights of paraffin section better with different types of tissue. Fatty tissue, such as breast tissue, sections very differently from muscle or gall bladder tissue. Because there are hundreds of different types of tissue and many sizes of cassettes and base molds the number of possible combinations can be very large. An automated system will manage the many possible permutations from, for example, lookup tables stored in each machine. A system which has many programmable variables, such as those which have access to an enhanced TSR, will allow the histotechnologist or smart processing machine to run selected optimum processing parameters for each cassette or other tissue carrier with the proper processing and embedding information to control each machine for precise sample preparation.

Various microtome sectionable tissue carriers are known. Sectionable tissue handling technology will evolve into different types of tissue immobilization. It is envisioned that these different types of the immobilization will require different tissue processing steps. Therefore, in addition to tissue sample information on the machine-readable indicator, specific tissue processing and embedding information may originate from a preprogrammed code associated with the type of cassette or other tissue sample immobilization device being used. Information stored in the TSR may pertain to the specific types of processing required for not only the tissue but the type of tissue immobilizing system used.

The availability of information about or associated with the particular sample should allow the embedding machine to determine how much paraffin should be dispensed based on the size and the quantity of tissue samples and cassettes. Other options for further automation and process control may become available when the type of sample is identified at the embedding center. For example, a particular sample may require vacuum assistance to pull very small air bubbles out of the paraffin, and this step could be automatically initiated upon instruction based on the information in TSR. For example, longer or shorter processing parameters can also be initiated based on the embedding material flow characteristics associated with any specific cassette or other immobilization device.

As the process continues on, each embedded cassette (or other immobilization device or tissue carrier) is then transferred to the microtomy station where the paraffin-embedded tissue samples and hardened embedding material (e.g., paraffin) are sectioned into thin slices to apply to microscope slides. Automated microtomy stations can also be enabled by having information which will be accessible to the microtome. This information may include certain parameters based on the sectionability of the particular cassette or tissue. Parameters such as the initial facing section thickness can be programmed to establish the precise thickness of material required to be removed prior to exposing and cutting the tissue. A smart microtome could read the DRC associated with the tissue carrier. As discussed previously, the DRC links to the TSR in the database which can then update the microtome with relevant parameters to be used during the microtome step or procedure. For example, these parameters may include the proper type of blade and blade or chuck settings for that particular type of tissue or cassette. Here again, the importance of understanding the prior embedding processes can be important. In the automated embedding center, the particular type of paraffin that was used for each individual tissue carrier device will be recorded. Different types of paraffin section better at different temperatures and with different types of grinds on the microtome blades. Therefore, by providing a traveling record (TSR) of the type of paraffin used for a particular sample, the automated microtomy center can adjust its parameters to section the tissue at a certain temperature, blade speed and section thickness, or indicate to the user a special blade and/or blade settings, or other parameters.

If equipped with a read/write memory device such as an RFID tag, a reader/writer will read a code on the cassette to access the TSR for that particular sample and, as the thin sections are transferred to slides, the slides will be given the same DRC as the associated tissue carrier. This will correlate each slide back to the information in the TSR associated with that sample, including all of the other associated process information as well as the patient information for that particular biopsy. In this way, there is a complete, unbroken chain of digital references built up in the TSR that extend from the original patient and the wrist band that was acquired at the time of patient registration through to microscopic diagnostic analysis by the pathologist.

To summarize, the process of biopsy sample retrieval through to diagnostic slide analysis currently involves many manual systems for recording the pertinent information that must follow along with a given tissue sample. Using a system and method such as outlined herein, all of the patient/billing information can be included along with any histopathology information to help define the particular processing required for the sample. Information useful for the pathologist at diagnosis can be tied to each sample with use of machine-readable indicators with or without reference to a separate database depending on the level of sophistication associated with the machine-readable indicator. The system, in any of its forms, allows a single TSR to be created and updated as the samples progress from retrieval to diagnosis. Below, a more detailed description of illustrative embodiments is given with reference to the drawings.

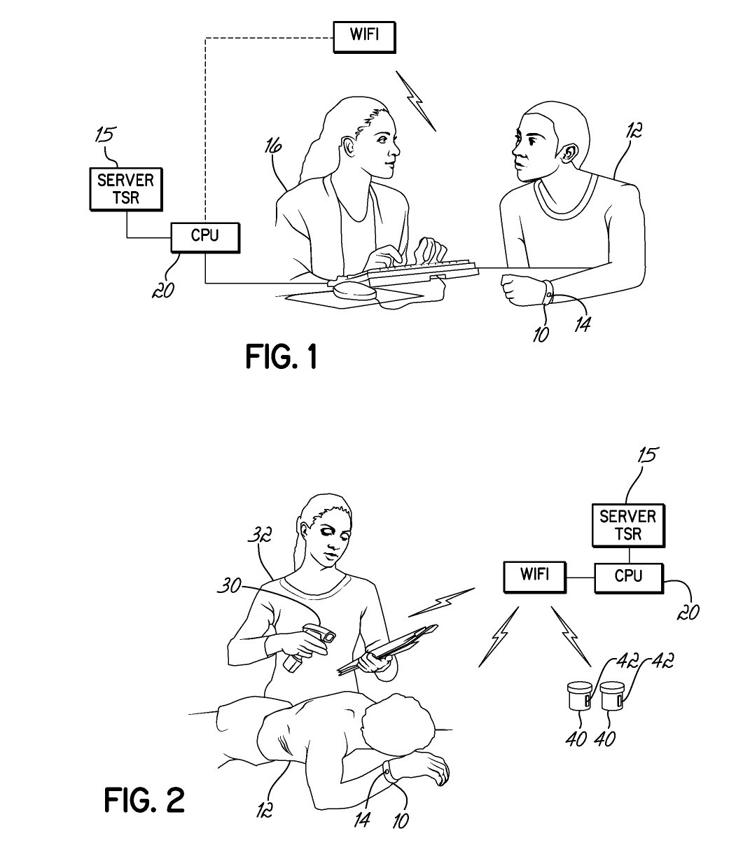

FIG. 1 illustrates an initiation of the TSR in patient admitting. A wrist band 10 is administered to each patient 12 upon admission to a hospital or other medical facility. This band 10 identifies the patient 12 and his doctor (not shown). The band 10 includes a barcode label or RFID tag 14, depending on the level of sophistication desired as discussed above, which assigns a unique reference, such as a Digital Reference Code (DRC) to the patient 12. In this example, an RFID tag 14 on the wrist band 10 is programmed with the patient's unique identifying DRC. This DRC can then be used to connect the patient's record or TSR 15 with each part of the diagnostic process. The RFID tag 14 may be programmed by a hospital employee 16, for example, using a computer 20 with WiFi or wireless capability. It will be appreciated that any of the data transmission occurring in the overall process described herein may be wireless, hardwired, or any combination thereof. The Internet and/or more localized intranets may be used for data transmission as well.

In admitting, the patient's record or TSR is initiated, associated with the DRC, and the TSR may receive patient information (name, address, and Social Security Number), insurance information, procedure date, as well as doctor and procedure detail information (e.g., doctor-referred testing and surgical procedures ordered). This establishes a tissue sample record (TSR) for the eventual diagnostic report.

Using a wireless interface or WiFi, with hardwired computer keyboard entry or a scanning device 30 and a computer 20, the admitting information or TSR will be transferred to a computer database 15 from which the information can be retrieved at any point further on in the biopsy sample processing.

FIG. 2 illustrates the step of the process occurring in, for example, a surgical suite. In the surgical suite, the identity of the patient 12 is confirmed via the wrist band 10, including the bar code or RFID tag 14 using a machine code reader or scanner 30. The nurse or medical professional 32 can record the patient's surgical information to the TSR server file or database 15 and can confirm what procedure is to be performed.

The particular surgical procedure(s) ordered is/are looked up in the TSR. The particular procedures to be performed will dictate which medical instruments will be used and what type and how many tissue samples will be retrieved or harvested from the patient 12. The portable reader/programmer 30 can take data from any instruments to be used by the surgeon which may trigger the need for specialized handling procedures later in the overall process and this information can then be made available to technicians or automated systems or machines, by linking with the particular TSR in the server database 15.

Biopsy sample containers 40 are labeled with the patient DRC, for example, using bar code labels or RFID tags 42. The TSR is updated with the type of patient tissue placed in the containers 40 it contains and how the tissue should be handled in later process steps. The DRC on each sample container 40 will uniquely identify the particular patient 12 and surgical procedure. This will allow for the tracking of all tissue samples taken from the patient 12 to begin as soon as they are placed into a container 40, even before reaching the pathology laboratory. The patient's TSR, such as in the database on server 15, will be updated to contain complete sample records, including the number of tissue samples harvested and how the samples were obtained. This information can later be recalled in the final diagnostic report.

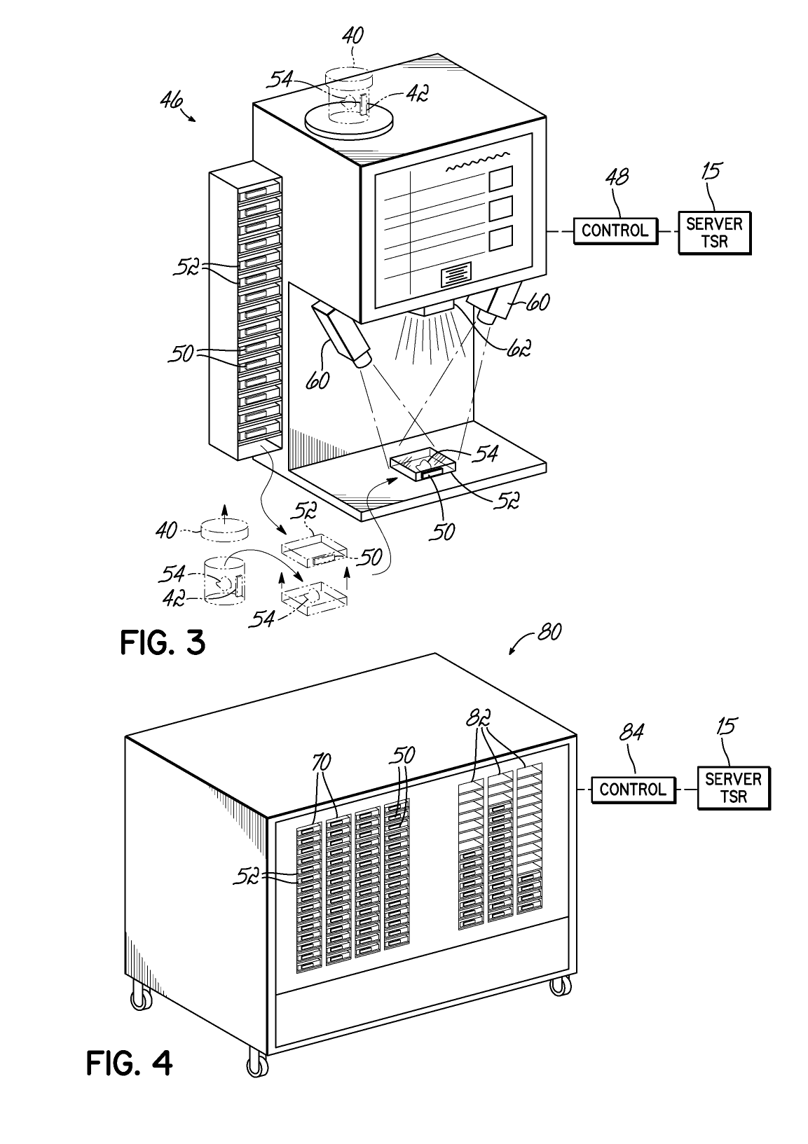

FIG. 3 illustrates the pathology lab gross-in step, including the use of a digital gross-in center or component 46. The biopsy containers 40 arrive at the pathology lab. In the pathology lab the sample containers 40 are processed through the digital gross-in center 46. Containers 40 are one of many types of tissue carriers that may be used in the overall histopathology process. Others include tissue cassettes, platforms or other structures used during the embedding procedure, and microscope slides used for examination/analysis purposes. The digital gross-in center 46 interacts with the TSR by wirelessly reading the DRC on the sample container 40 both to retrieve information about the sample from the TSR 15 and to record any new information to be used further on in the process either by a technician or other professional, or by an automated machine.

The digital gross-in center 46 includes a reader whereby the particular sample container 40 is specifically identified to the gross-in center 46. The gross-in center 46 programs a machine-readable indicator, such as an RFID tag 50, secured to a tissue carrier such as a cassette 52 into which the tissue sample 54 is transferred from one of the containers 40. The programming function may be achieved using a suitable electronic control 48, such as a computer or other electronic processor. The other electronic controls associated with other histological processing equipment disclosed herein may likewise be comprised of computerized controls or electronic processors depending on the capabilities needed. A stereo digital camera 60 allows for 3D imaging or an infrared camera and light 62, microwave, ultrasonic, or other type of imaging system records an image of the tissue 54 as it appears on the cassette 52 or other tissue carrier, giving a physical record of the tissue sample 54 before it is sectioned. This gives the TSR 15 an archival image of the tissue, including the number of samples and their appearance, as associated with the tissue carrier 52, for later reference and verification purposes. This may also allow a calculation of the volume of the sample(s) 54 to be made which can be used to specify the length of processing time required in a tissue processing machine, the volume of embedding material needed per sample and/or overall paraffin volume requirements during an automated embedding process, or other variables. Determination of the volume of embedding material (e.g., paraffin) needed for each tissue sample or samples associated with many cassettes being run through an embedding machine will provide for automated inventory control of the embedding material supplied to the embedding machine. If the type of tissue is identified by the pathologist and recorded in the TSR 15, an automatic lookup table could be used to determine the density of the tissue sample 54. This information, along with other input or measured parameters, can be used to assign a processing code to each tissue sample 54 by means of a computer algorithm. This will ensure that, for example, a small, dense tissue like prostate tissue is not assigned the same processing code as a much larger piece of liver which is less dense. So, like or similar tissue types may be able to be batched more efficiently for processing. For example, like tissue types may use like processing reagents and processing cycle times, and also like types of paraffin. The processing efficiency will be improved by the ability to use all of the sample information in conjunction with a computer program instead of manually batching like tissue samples.

The image taken of each sample 54 during gross-in can also be recalled from the TSR 15 on the server at the point of creating the final report to take the place of or to supplement a written physical description of the sample 54, simplifying that process and making it more complete.

The digital gross-in center 46 can also voice record a traditional physical description by the pathologist, saving the information as a .wav file which can then be automatically transcribed into the TSR 15 by transcription software or by a transcriptionist. This continues to build the digital record of information on the TSR server 15 and the information is available at the end of the processing to aid in producing the final report.

The gross-in center 46 also allows the pathologist to add particular instructions to the machine-readable indicator 50 on the cassette 52 for the tissue processing center, the embedding center and/or the staining center, for example, used later in the process to ensure that particular features of interest in the tissue 54 are best visualized for diagnosis. This gives the pathologist the opportunity to handle each tissue sample 54 individually and to ensure that the appropriate procedures are performed to obtain the best diagnostic information possible. For example, a pathologist may take a single sample 54, cut it into multiple pieces, and specify different procedures and/or stains to be applied to each separate piece or sample further on in the process to enhance different features in the sample. No further human interaction would be necessary to differentiate the required treatment of each sample since the instructions for further processing are programmed into the cassette RFID tag 50 or retrieved from the TSR 15.

The gross-in center 46 can also enable efficiency tracking of workers. By requiring each employee to enter a work number at the start of their session, productivity can be monitored based on the throughput of samples. Subroutines can be run on all the TSR's stored on the server 15 and comparisons made on the time required to complete a step or procedure in the histopathology process. This is true for any of the procedures discussed herein including, for example, microtomy where human efficiency is an important factor in productivity. This enables work flow analysis and cost comparisons for billing and audit purposes. Predictive models can then be used to manage material usage and implement programs to increase work flow efficiencies, such as through studies of any parts of the overall process that present slowdowns or bottlenecks.

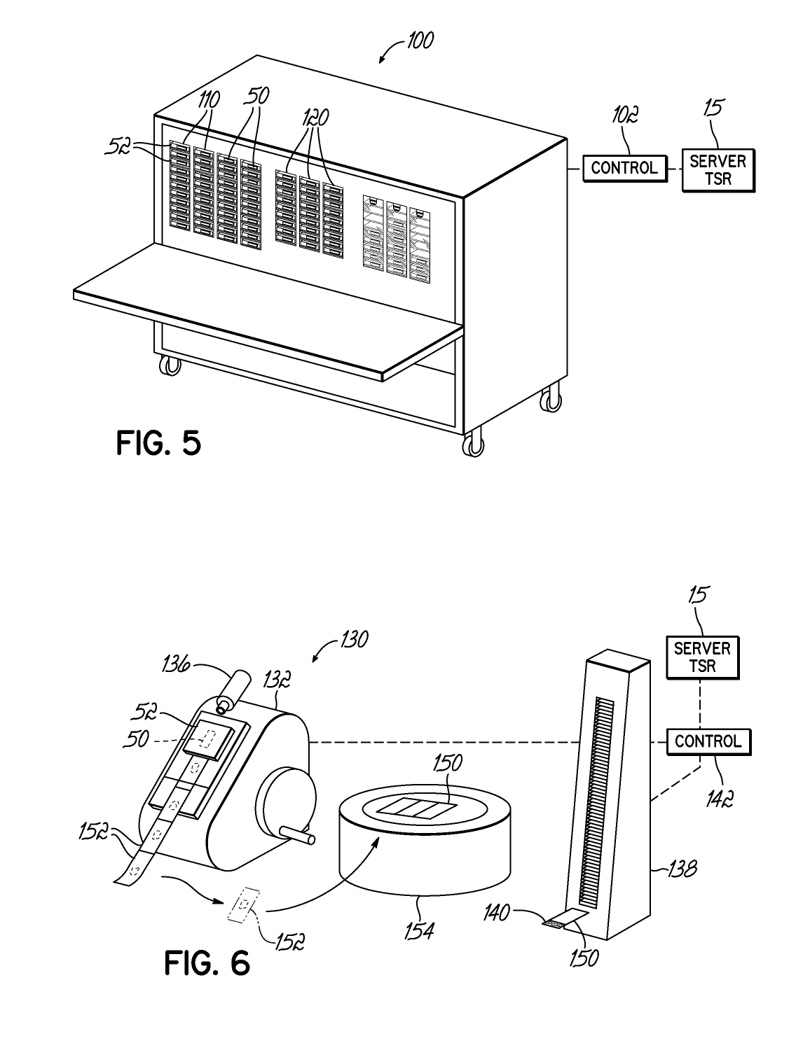

FIG. 4 illustrates a tissue processing procedure. After gross-in, the cassettes 52 are inserted randomly into racks 70, to await processing. Tissue processing is often referred to as tissue fixation or fixing. It is the step of preparing the tissue for embedding such that the tissue may be infiltrated with paraffin. Ideally, the tissue sample(s) is/are then embedded in a block of paraffin that will have similar density and cutting characteristics across its entire cross section, including the portion containing the sample(s). The tissue embedding procedure is discussed later in connection with FIG. 5. A tissue processing machine, sometimes referred to as a tissue processor 80, can query each cassette 52 by reading the DRC, i.e., bar code or RFID tag 50 and then accessing the TSR 15 for that tissue sample in the server. A suitable electronic control 84 may be used in connection with the processor 80 to facilitate the reading, accessing and other necessary processing functions. The TSR contains data required to determine the best processing cycle for that particular sample—specified by cassette size, sample thickness, required reagent or temperature, among other parameters which were added to the TSR 15 at the digital gross-in center 46 or prior to that in the case of a surgical issue. The processor 80 can then determine which samples to batch together in separate groups 82 and what type of processing cycle to run. The processor, or processing machine 80, will be able to pick and place each cassette 52 into a processing chamber according to its processing parameters. In this manner, the cassettes 52 are processed in efficient groups saving processor reagents and processing time. In addition, tissue samples which need to be processed in an expedited manner, for a rush order, could be singled out automatically by the machine 80 and handled appropriately. This provides an efficient, automated process, without requiring significant human intervention or management.

The tissue processor 80 can write data to the TSR 15 regarding how each tissue sample was processed. The final report is thereby assured to contain the correct information on how a particular sample was handled throughout the process. In such a system with onboard computers, the processor 80 can also keep track of how many cassettes 52, and what type of cassettes 52, were processed. This can be useful for accounting or ordering of supplies. The system also allows the processor 80 to track each reagent used and suggested maintenance based on actual usage.

FIG. 5 illustrates an automated embedding center 100 and its use. Once processed, each cassette 52 must be embedded in paraffin. The cassettes 52 are again randomly inserted into racks 110 to await embedding in the automated embedding center 100, such as the Auto TEC Embedding Center by Sakura or as described in U.S. Patent Publication No. 2005/0226770, the disclosure of which is incorporated by reference herein. Using a suitable electronic control 102, the embedding center 100 can query each cassette 52 by reading the DRC associated with the RFID tag 50 or other machine-readable indicator (and accessing the TSR 15 to determine the required embedding parameters, such as size of the embedding mold, required embedding material (paraffin parameters), priority of the sample, etc.). The cassettes 52 currently have physical query points which can be read to determine the size of embedding mold required. More detailed process specifications can be linked to the specific sample via the TSR 15 or RFID chip 50 on the cassette 52 and the TSR 15 is automatically updated. The embedding center 100 then can automatically batch cassettes 52 with like processing parameters in groups 120 or reconfigure the processing parameters for each individual cassette 52 after reading its RFID tag 50 and corresponding TSR 15. The cassette 52 is embedded in paraffin or other embedding material.

The embedding center 100 updates each TSR 15 with information establishing how each sample was processed. The final report is assured to contain the correct information for how a particular sample was handled throughout the embedding process.

FIG. 6 illustrates a microtomy center 130 and its use. A microtome device 132 reads the indicator 50 on each individual cassette 52 with, for example, an electronic optical or RF reader 136 and sends identification information to a programming device 138 which appropriately records the DRC on machine-readable indicators 140 on microscope slides 150 which receive sections from the cassette 52. A suitable electronic control 142 is used to facilitate the necessary exchange of information, for example, between the reader 136 and device 138, and also the exchange of information with the TSR 15, as in other portions of the overall procedure. The microtome device 132 creates thin ribbon sections 152 from the cassette 52 which are then floated onto the microscope slides 150 in a water bath 154. The RFID tag or other indicator 50 on the cassette 52 remains embedded in the paraffin block from which the sections are taken. This block is then archived and can be recalled (by electronic RFID query in the block storage facility) should further sections be required for later examination. The archive location can be recorded in the TSR on the server 15. The microscope slides 150 are labeled with the cassette/paraffin block DRC via the RFID tag 140 so traceability to the original tissue sample TSR 15 is assured. This provides access to the information originating from the original gross-in of the sample and, for example, specifying what type of staining is required for the tissue sample. A record of the number of microscope slides created from each cassette/paraffin block is created in the TSR 15 for traceability.

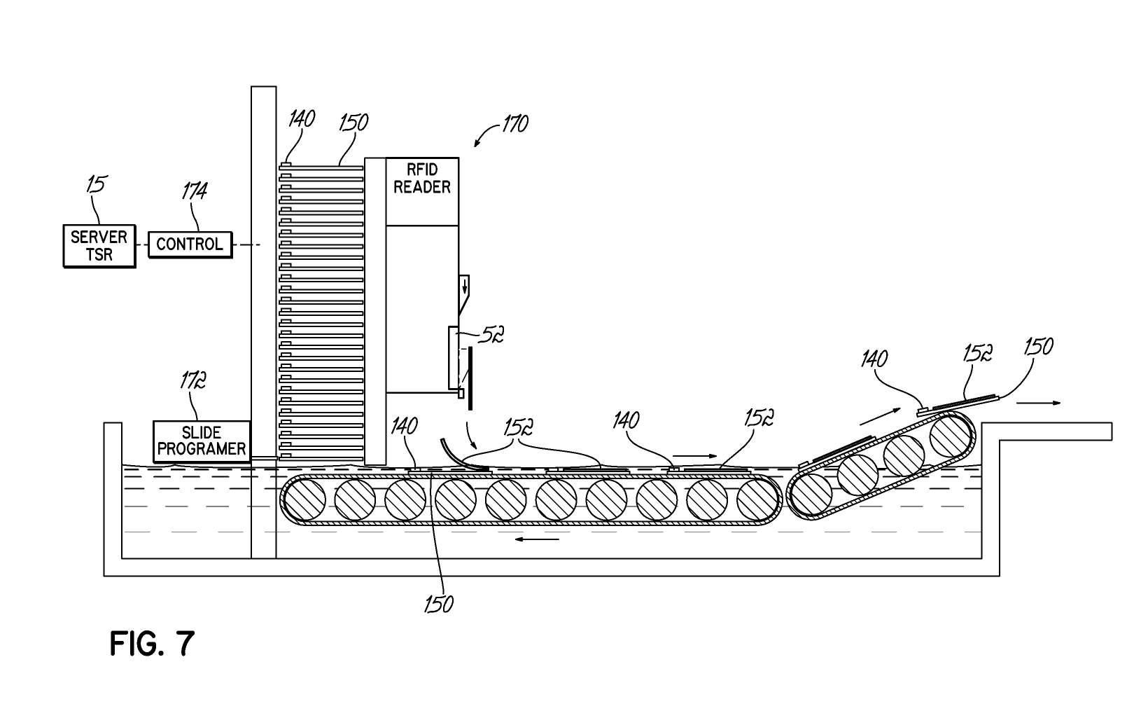

FIG. 7 illustrates an alternative automated microtomy and microscope slide preparation center or component 170 and its use. This component 170 automates the processes described in FIG. 6. Using the information retrieved from the machine-readable indicator 50 on the cassette/paraffin block, the slide preparation center 170 automates the process of receiving the cassette/paraffin block 52, sectioning it and applying the ribbon sections 152 to appropriately labeled microscope slides 150 with associated machine-readable indicators 140. Indicators 140 are encoded by use of a programmer 172, for example, to have DRC's matching with the corresponding tissue sample. As in other apparatus disclosed herein, a suitable electronic control 174 (such as a computer or electronic processor) is used to facilitate exchange of information and performance of control functions.

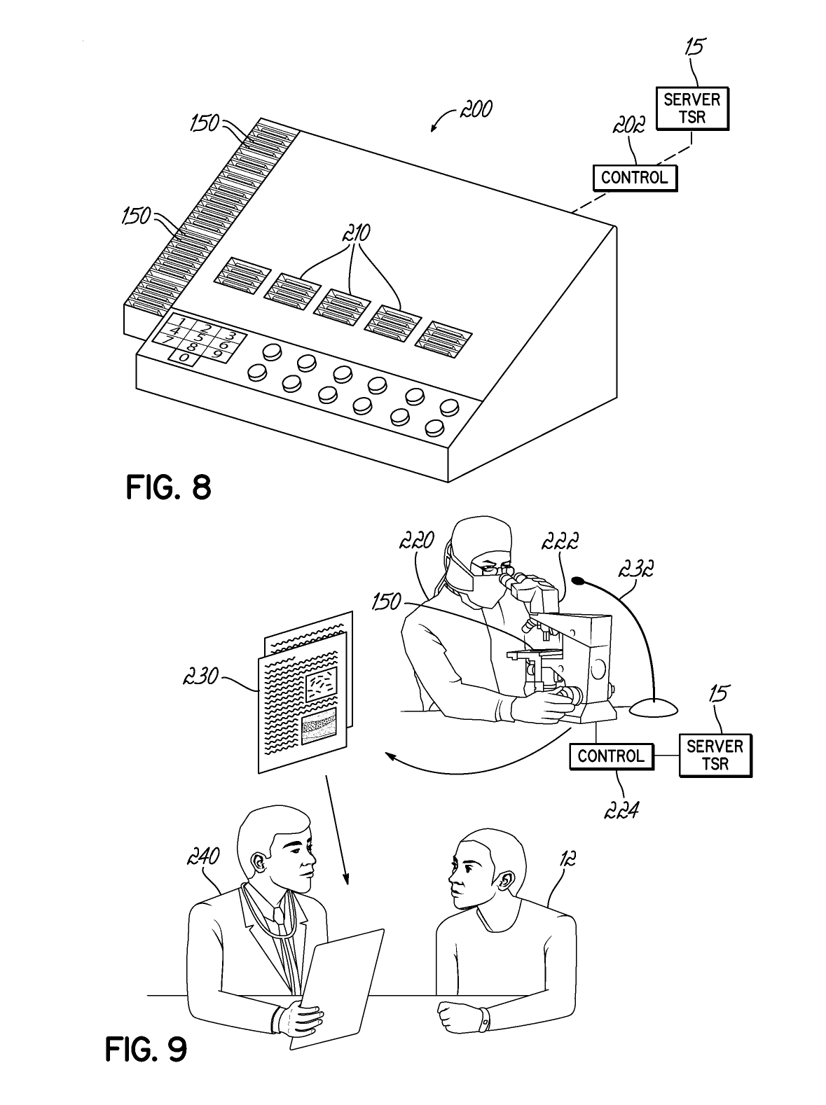

FIG. 8 illustrates a staining center 200 and its use. The microscope slides 150 are inserted in random order into the staining center 200. Using the information obtained through reading the DRC from the indicator 140 on each slide 150 and retrieving data from the TSR 15, via an electronic control 202, the staining center 200 can pick and place the slides 150 to batch those with similar processing parameters in groups 210. These parameters can include common stain sequences, timing, priority, etc. The server record or database 15 for the patient is updated with information confirming that each slide 150 has been processed through this center 200. Information is transferred to the TSR 15 which will be used for billing purposes, for example, including the number of slides prepared and any special procedures performed.

The staining center 200 sorts the slides 150 into appropriate slide trays per patient for review by the pathologist. With the ability to track the total number of slides 150 created per patient (from information already recorded in the patient's record), the sorter can identify which trays are complete and ready for review while others await process completion of all slides.

FIG. 9 illustrates pathologist review and final report generation. The pathologist 220 reviews the slides 150 under a microscope 222. The microscope 222 can include an electronic control 224, or the control 224 may be a separate component. In either case, it may perform functions related to the exchange of information and/or control functions of the procedure as described herein. Using a reporting system, he or she can recall all of the information that has been stored from patient admitting through the sample retrieval in the surgical suite and the slide preparation process. He can verify proper identification and handling of the samples. Standardized report forms 230 contain information blocks and digital picture links which automatically fill in based on the DRC recorded on the indicator 140 carried on the microscope slides 150. A digital record of the microscopic analysis of the slide 150 can be recorded as well. Additional information and diagnosis can be added to the report and TSR 15 by dictation using a microphone 232, and subsequent automated transcription and/or manual keyboard entry. The pathologist 220 describes his/her analysis using the microphone 232, for example, which may be recorded as an audio file, such as a .wav file. This file may be saved as an audio file and/or a text file in the TSR 15. The microscope 222 preferably has the ability to allow the pathologist to take digital photographic images of the tissue sample(s), and these images may be made part of the pathology report or otherwise part of the TSR 15. This digital record can then be electronically transmitted back to the patient's physician 240.

TABLE SUMMARY OF PROCESS STEPS

| | | |
|---|---|---|
| Procedural | Digital Reference Code | Tissue Sample Record |
| step | (DRC): | (TSR): |
| | Unique identification code | Cumulative data on server or |
| | (e.g., number) for each | database for each case or |
| | tissue harvesting event | sample |
| | requiring a report | |
| Admitting | Unique Patient Tissue | All admitting records |
| | Identifier established with | Billing information (e.g., |
| | the DRC | insurance) |
| | (Wrist band ID RFID could | Establishes unique case |
| | include the DRC) | code (Tissue Sample Record |
| | | is established in the |
| | | Database referenced to the |
| | | unique DRC) |
| Surgery | Sample container(s) each | TSR updated with: # of |
| | carry the DRC to identify | samples in container(s); |
| | that patient's sample(s) | type of biopsy samples |
| | | special processing codes; |
| | | instrument used to take |
| | | samples; |
| | | urgency codes; special |
| | | disease suspected; etc. |
| Tissue | Sample container(s) | TSR updated-Update |
| transport | Time out of surgery, | container(s) record with time |
| to lab | Courier identifier, etc. | out of surgery, time logged |
| | (RFID or bar code on | into hospital receiving, etc. |
| | container identifies DRC | |
| | unique sample #) | |
| Tissue | Sample container(s) | TSR updated - Update |
| logged in | (RFID or bar code on | container(s) record with time |
| at lab | container identifies DRC | into pathology lab |
| | unique sample #) | |
| Tissue | Tissue carrier Devices | TSR updated - Cassette |
| grossed in | (Cassette(s)) programmed | DRC's recorded 3D gross-in |
| | with DRC | record (program runs |
| | Histopathology case code | process calculation, mass |
| | Special processing bits | and density surface area of |
| | encoded for: | sample) Visual record |
| | Tissue processing, | Dictation files recorded |
| | Embedding, | Cassette file recorded |
| | Microtomy, | Special processing codes |
| | Slide staining, | recorded to tell tissue |
| | Embedding mold | processor how to batch like |
| | Other steps | samples. |
| Tissue | Processing machine reads | TSR updated - Processor |
| processor | DRC(unique sample #) on | updates TSR when each |
| | tissue carrier(s) then | Cassette is processed. |
| | accesses TSR and tissue | Batches configured from like |
| | processed (i.e., fixed) | processing parameters and |
| | according to instructions or | sorted by batch, case or |
| | programs from TSR | other priority. |
| | | Error codes, reagents low, |
| | | contaminated etc. |
| | | Time in/out of processor |
| | | uploaded to TSR. |
| Embedding | Embedding machine reads | TSR updated - Embedding |
| center | DRC(unique sample #) on | center updates TSR to |
| | tissue carrier(s) then | include embedding records. |
| | accesses TSR and tissue | Cassettes in machine |
| | embedded according to | queried and sorted by batch, |
| | instructions or programs | case or other priority, e.g., |
| | from TSR | type of paraffin, |
| | | mold/base size used |
| Microtomy | Microtome machine reads | TSR updated - Microtomy |
| | DRC(unique sample #) on | center updates TSR to |
| | tissue carrier(s) then | include embedding records. |
| | accesses TSR and tissue | Special stains used on |
| | sliced according to | slides, number of slides |
| | instructions or programs | created, and slide records |
| | from TSR | initiated |
| Slide Stainer | Staining machine reads | TSR updated - TSR is |
| | DRC(unique sample #) on | updated with slide |
| | tissue carrier(s) then | information and process |
| | accesses TSR and tissue | parameters used |
| | stained according to | |
| | instructions or programs | |
| | from TSR | |
| Pathology | Pathology report center | TSR updated - Sample |
| report | reads DRC(unique sample | records updated with report |
| | #) on slides(s) then | information |
| | accesses TSR and report | Billing information output on |
| | is linked to the TSR | all materials used in |
| | | processes |
| Archives | Slides and cassettes are | TSR updated - Location of |
| | put into storage archive. | cassettes and slides in |
| | DRC (e.g., RFID) can be | permanent storage on TSR |
| | read to retrieve for | |
| | consultation | |

While the present invention has been illustrated by a description of various preferred embodiments and while these embodiments have been described in some detail, it is not the intention of the Applicant to restrict or in any way limit the scope of the appended claims to such detail. Additional advantages and modifications will readily appear to those skilled in the art. The various features discussed herein may be used alone or in any combination depending on the needs and preferences of the user. This has been a description of illustrative aspects and embodiments the present invention, along with the preferred methods of practicing the present invention as currently known. However, the invention itself should only be defined by the appended claims.

What is claimed is:

1. A system for processing tissue biopsy samples during a histopathology process, comprising:
a tissue carrier constructed to carry a tissue biopsy sample during at least one step of the histopathology process;
a database storing information associated with the tissue biopsy sample and associated with a tissue processing procedure;
a machine-readable indicator physically associated with the tissue carrier, the machine-readable indicator including a machine-readable reference identified with the tissue biopsy sample; and
an electronic control operative to read the reference, access the information in the database using the reference, and implement at least a portion of the tissue processing procedure in accordance with the accessed information.

2. The system of claim 1, further comprising:
a tissue processing device including the electronic control and operative to carry out at least a portion of the tissue processing procedure.

3. The system of claim 2, wherein the tissue processing device is selected from the group consisting of:
a tissue gross-in device, a tissue processor, a tissue embedding device, a tissue microtomy device, a tissue slide preparation device, and a diagnostic slide reading device.

4. The system of claim 1, wherein the electronic control is operative to update the information in the database.

5. The system of claim 1, wherein the machine-readable indicator stores information for use in establishing processing parameters of tissue biopsy sample.

6. The system of claim 5, wherein the electronic control is operative to update the information stored on the machine-readable indicator.

7. The system of claim 6, wherein the machine-readable indicator further comprises an RFID tag.

8. The system of claim 1, wherein the reference further comprises a digital reference code.

9. The system of claim 1, wherein the machine-readable indicator is a read-only indicator.

10. The system of claim 9, wherein the read-only indicator further comprises a bar code or an etching on the tissue carrier.

11. A system for processing tissue biopsy samples during a histopathology process, comprising:
a tissue carrier constructed to carry a tissue biopsy sample during steps of the histopathology process;
a machine-readable and writable indicator physically associated with the tissue carrier, the machine-readable and writable indicator storing a reference identified with the tissue biopsy sample, and further storing information associated with at least one physical attribute of the tissue biopsy sample; and
an electronic control operative to read the reference and the information stored on the machine-readable and writable indicator, and implement at least a portion of the tissue processing procedure in accordance with the accessed information.

12. The system of claim 11, wherein the electronic control is further operative to write new information on the machine-readable and writable indicator.

13. The system of claim 12, wherein the new information comprises updating of the information already stored on the machine-readable and writable indicator.

14. The system of claim 11, wherein the information stored on the machine-readable and writable indicator is identical to at least a portion of information stored in a separate database.

15. The system of claim 11, wherein the information stored on the machine-readable and writable indicator is at least partially different than the information stored in the database.

16. The system of claim 11, further comprising:
a tissue processing device including the electronic control and operative to carry out at least a portion of the tissue processing procedure.

17. The system of claim 16, wherein the tissue processing device is selected from the group consisting of:
a tissue gross-in device, a tissue processor, a tissue embedding device, a tissue microtomy device, a tissue slide preparation device, and a diagnostic slide reading device.

18. The system of claim 11, wherein the electronic control is operative to update the information in the database.

19. The system of claim 11 wherein the machine-readable indicator stores information for use in establishing processing parameters of tissue biopsy sample.

20. The system of claim 19, wherein the electronic control is operative to update the information stored on the machine-readable indicator.

21. The system of claim 20, wherein the machine-readable indicator further comprises an RFID tag.

22. The system of claim 11, wherein the reference further comprises a digital reference code.

23. A system for embedding tissue biopsy samples during a histopathology process, comprising:
a tissue carrier constructed to carry a tissue biopsy sample during an embedding procedure;
an embedding device operative to embed the tissue biopsy sample for subsequent microtomy;
a database storing information associated with the embedding process;
a machine-readable indicator physically associated with the tissue carrier, the machine-readable indicator including a machine-readable reference identified with the tissue biopsy sample; and
an electronic control operatively coupled with the embedding device and capable of reading the reference and the information stored on the machine-readable indicator, accessing the information in the database, and implementing at least a portion of the embedding procedure in accordance with the information.

24. The system of claim 23, wherein the information stored in the database is used to direct the embedding device to place the tissue biopsy sample into one of at least two different molds for receiving the embedding material.

25. The system of claim 23, wherein the information is stored in the machine-readable indicator and is used to direct the embedding device to place the tissue biopsy sample into one of at least two different molds for receiving the embedding material.

26. The system of claim 23, wherein the information stored in the database is used to direct the embedding device to heat and/or cool embedding material used for embedding the tissue biopsy sample.

27. The system of claim 23, wherein the information is stored in the machine-readable indicator and is used to direct the embedding device to heat and/or cool embedding material used for embedding the tissue biopsy sample.

28. The system of claim 23, wherein the information stored in the database is used to select an embedding material for embedding the tissue biopsy sample.

29. The system of claim 23, wherein the information is stored in the machine-readable indicator and is used to select an embedding material used for embedding the tissue biopsy sample.

30. The system of claim 23, wherein the electronic control is operative to update the information in the database.

31. The system of claim 23, wherein the electronic control is operative to update the information stored on the machine-readable indicator.

32. The system of claim 23, wherein the machine-readable indicator further comprises an RFID tag.

33. The system of claim 23, wherein the reference further comprises a digital reference code.

34. A system for processing tissue biopsy samples during a histopathology process, comprising:
a tissue carrier constructed to carry a tissue biopsy sample during a procedure for enabling subsequent embedding of the tissue biopsy sample;
a tissue processor operative to subject the tissue biopsy sample to the procedure for enabling subsequent embedding of the tissue biopsy sample;
a database storing information associated with the procedure;
a machine-readable indicator physically associated with the tissue carrier, the machine-readable indicator including a machine-readable reference identified with the tissue biopsy sample; and
an electronic control operatively coupled with the tissue processor and capable of reading the reference, accessing the information in the database using the reference, and implementing at least a portion of the procedure in accordance with the information.

35. The system of claim 34, wherein the information stored in the database is used to direct the tissue processor with respect to at least one of: chemical reagents, cycle times or cycle temperatures used during the procedure.

36. The system of claim 34, wherein the information is stored in the machine-readable indicator and is used to direct the tissue processor with respect to at least one of: chemical reagents, cycle times or cycle temperatures used during the procedure.

37. The system of claim 34, wherein the electronic control is operative to update the information in the database.

38. The system of claim 37, wherein the database is on the machine-readable indicator.

39. The system of claim 34, wherein the machine-readable indicator further comprises an RFID tag.

40. The system of claim 34, wherein the reference further comprises a digital reference code.

41. A system for carrying out at least a portion of a histopathology process, comprising:
a tissue carrier constructed to carry a tissue biopsy sample;
a machine-readable indicator physically associated with the tissue carrier, the indicator providing a machine-readable reference identified with the tissue biopsy sample; and
a database storing a tissue sample record associated with the reference of the machine-readable indicator, whereby information in the tissue sample record is usable to assist with the performance of at least one step in the histopathology process of the biopsy tissue sample.

42. The system of claim 41, wherein the information in the tissue sample record is usable to assist in the performance of at least one of: processing of the tissue biopsy sample prior to embedding of the tissue biopsy sample, embedding of the tissue biopsy sample, microtomy slide preparation of the tissue biopsy sample, staining of the tissue biopsy sample on a slide, or preparation of a final pathology report on the tissue biopsy sample.

43. A system for processing tissue biopsy samples during a histopathology process, comprising:
a plurality of tissue carriers each constructed to carry a tissue biopsy sample during the histopathology process;
a machine operative to carry out at least one procedure during the histopathology process;
a database storing information associated with the procedure;
a plurality of machine-readable indicators respectively physically associated with the tissue carriers, each machine-readable indicator including a unique machine-readable reference identified with the tissue biopsy sample associated with the corresponding tissue carrier; and
an electronic control operatively coupled with the machine and capable of reading the references accessing the information in the database using the unique references, and implementing at least a portion of the procedure based on like information stored with respect to the different references.

44. The system of claim 43, wherein the electronic control excludes at least one operation within the procedure from being performed on at least one of the tissue biopsy samples based on the like information.

45. The system of claim 44, wherein the electronic control includes at least one operation within the procedure to be performed on at least one of the tissue biopsy samples based on the like information.

46. The system of claim 44, wherein the electronic control is operative to update the information in the database.

47. The system of claim 44, wherein the electronic control is operative to update the information stored on the machine-readable indicator.

48. The system of claim 44, wherein the machine-readable indicator further comprises an RFID tag.

49. The system of claim 44, wherein the reference further comprises a digital reference code.

50. A method of processing tissue biopsy samples during a histopathology process, comprising:
reading a machine-readable indicator physically associated with a tissue carrier to obtain a reference associated with a tissue biopsy sample carried by the carrier;
accessing information in a database associated with the reference obtained from the machine-readable indicator; and
performing at least procedure during the histopathology process using the information.

51. The method of claim 50, wherein a tissue processing device includes a reader and the reader is used to read the machine-readable indicator, the method further comprising:
performing the procedure with the tissue processing device.

52. The method of claim 51, wherein the tissue processing device is selected from the group consisting of:
a tissue gross-in device, a tissue processor, a tissue embedding device, a tissue microtomy device, a tissue slide preparation device, and a diagnostic slide reading device.

53. A method of processing tissue biopsy samples during a histopathology process, comprising:
reading a machine-readable indicator physically associated with a tissue carrier to obtain information associated with the tissue biopsy sample carried by the tissue carrier; and
performing at least one procedure during the histopathology process using the information.

54. The method of claim 53, wherein a tissue processing device includes a reader and the reader is used to read the machine-readable indicator, the method further comprising:
performing the procedure with the tissue processing device.

55. The method of claim 54, wherein the tissue processing device is selected from the group consisting of:
a tissue gross-in device, a tissue processor, a tissue embedding device, a tissue microtomy device, a tissue slide preparation device, and a diagnostic slide reading device.

56. The method of claim 53, wherein the step of performing the procedure further comprises at least one of the following steps:
a) using the information to select a parameter of a tissue gross-in procedure for recording information associated with the tissue biopsy sample;
b) using the information to select a parameter of a tissue fixation procedure for preparing the tissue biopsy sample for embedding;
c) using the information to select a parameter of a tissue embedding procedure for preparing the tissue biopsy sample for microtomy and microscope slide preparation; and/or
d) using the information to select a parameter of a microtomy procedure for preparing microscope slides of the tissue biopsy sample.

57. A method of processing tissue biopsy samples during a histopathology process, comprising:
reading a machine-readable indicator physically associated with a tissue carrier to obtain information associated with the tissue biopsy sample carried by the tissue carrier;
accessing an electronic tissue sample record based at least partially on the information; and
controlling at least one procedure during the histopathology process using the accessed information, the procedure selected from the group consisting of: tissue fixation, tissue embedding, microtomy, microscope slide preparation, microscope slide staining, and preparation of a pathology report.

58. The method of claim 57, further comprising:
updating the information in the electronic tissue sample record with information for use in subsequent procedures of the histopathology process.

59. The method of claim 57, wherein controlling at least one procedure further comprises:
making inclusionary or exclusionary batch decisions based on a comparison of like or similar parameters associated with different tissue carriers and/or different tissue biopsy samples.

60. The method of claim 57, wherein the information contained on the machine-readable indicator includes a digital reference code associated with at least one of the tissue biopsy sample or the tissue carrier, and the electronic tissue sample record is at least partially stored in a database remote from the machine-readable indicator.

61. The method of claim 60, wherein the electronic tissue sample record is at least partially contained in a database stored in a control of a device used in the histopathology process.

62. The method of claim 57, wherein the electronic tissue sample record is at least partially stored on the machine-readable indicator.

63. The method of claim 57, wherein the machine-readable indicator only contains a digital reference code and other information of the electronic tissue sample record is stored in a database remote from the machine-readable indicator.
